(19) United States
(12) Patent Application Publication
(10) Pub. No.: US 2010/0092495 A1
(43) Pub. Date: Apr. 15, 2010

(54) POTENT CELL-BINDING AGENT DRUG CONJUGATES

(75) Inventor: Ravi V. J. CHARI, Newton, MA (US)

Correspondence Address: SUGHRUE MION, PLLC, 2100 PENNSYLVANIA AVENUE, N.W., SUITE 800, WASHINGTON, DC, 20037, US

(73) Assignee: IMMUNOGEN INC., Waltham, MA (US)

(21) Appl. No.: 12/574,430

(22) Filed: Oct. 6, 2009

Related U.S. Application Data
(63) Continuation-in-part of application No. 12/433,638, filed on Apr. 30, 2009, now abandoned.
(60) Provisional application No. 61/049,296, filed on Apr. 30, 2008.

(51) Int. Cl. A61K 39/395; C07K 16/18; A61P 35/04

(52) U.S. Cl. 424/181.1; 530/391.1

(57) ABSTRACT

The present invention relates to the use of about 2 to about 8 drug molecules, for example, maytansinoid, per cell binding agent, such as an antibody, and maximal efficacy as compared to a drug load of lesser or higher number of drugs linked to such a cell binding agent.

This is a continuation-in-part of United States Non-Provisional application Ser. No. 12/433,638, filed Apr. 30, 2009, which claims priority to U.S. Provisional Application No. 61/049,296, filed Apr. 30, 2008. The entire disclosures of the prior applications, application Ser. Nos. 12/433,638 and 61/049,296 are considered part of the disclosure of the accompanying continuing application and are hereby incorporated by reference.

### FIELD OF INVENTION

The present invention relates to efficacious drug load in a drug cell-binding agent conjugate. In particular, the present invention relates to the use of about 2 to about 8 drug molecules (drug load), for example, maytansinoid, per cell binding agent, such as an antibody, and their greater efficacy as compared to a drug load of a lesser or higher number of drugs linked to such a cell binding agent.

### BACKGROUND

A major drawback with existing drug cell-binding agent conjugates is their inability to deliver a sufficient concentration of drug to the target site because of the limited number of targeted antigens and the relatively moderate cytotoxicity of cancer drugs like methotrexate, daunorubicin, maytansinoids, taxanes and vincristine. In order to achieve significant cytotoxicity, linkage of a large number of drug molecules either directly to the antibody or through a polymeric carrier molecule becomes necessary. However such heavily modified antibodies often display impaired binding to the target antigen and fast in vivo clearance from the blood stream. Therefore, there is a need to improve the ability to deliver a sufficient concentration of a drug to the target such that maximum cytotoxicity for the drug is achieved.

### SUMMARY OF INVENTION

Accordingly, the present invention provides a drug cell-binding agent conjugate wherein the number of drug molecules per cell-binding agent molecule is about 2 to about 8.

This invention also encompasses the use of drug molecules (e.g., maytansinoids), as an active ingredient for the manufacture of drug cell-binding agent conjugates wherein the number of drug molecules per cell-binding agent molecule is about 2 to about 8

In another aspect, the cell-binding agent drug conjugate is represented by formula (D)ₙ-L-CBA, wherein D is a drug, n is about 2 to about 8, L is a linker, wherein the linker is selected from a cleavable linker or a linker substantially resistant to cleavage, and CBA is a cell binding agent.

In a preferred element, the drug cell binding agent conjugate is represented by formula (May)ₙ-L-CBA, wherein May is a maytansinoid, n is about 2 to about 8, L is a linker, wherein the linker is selected from a cleavable linker or a linker substantially resistant to cleavage, and CBA is a cell binding agent.

### FIGURES

FIG. 1 illustrates the anti-tumor effect of C242-DM1 conjugate containing variable number of DM1 molecules/C242 molecule.

FIG. 2. Structural representation of huN901-DM1 conjugates.

FIG. 3. Binding of huN901 antibody and huN901-DM1 conjugates.

FIG. 4. In vitro cytotoxicity of huN901-DM1 bearing 1.0 to 6.6 DM1/Antibody.

FIG. 5. In vivo antitumor activity of huN901-DM1 conjugates bearing 1.0 to 6.6 DM1/Antibody.

### DETAILED DESCRIPTION

The present invention is based on the unexpected finding that when about 2 to about 8 drug molecules (“drug load”), for example, maytansinoid, are linked to a cell binding agent, such as an antibody, the anti-tumor effect of the conjugate is much more efficacious as compared to a drug load of a lesser or higher number of drugs linked to the same cell binding agent.

“Drug load”, as used herein, refers to the number of drug molecules (e.g., a maytansinoid) that can be attached to a cell binding agent (e.g., an antibody). In one aspect the number of drug molecules that can be attached to a cell binding agent may average from about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1). In a preferred aspect, the number of drug molecules that can be attached to a cell binding agent may average from about 2 to about 7 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1). In an even more preferred aspect the number of drug molecules that can be attached to a cell binding agent may average from about 2 to about 6 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1). In the most preferred embodiment, the number of drug molecules that can be attached to a cell binding agent may average from about 2 to about 5 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 5.1). Examples of suitable ranges are where n is about 2.5 to about 6.6, when n is about 2.5 to about 5.8, and when n is about 2.5 to about 4.5.

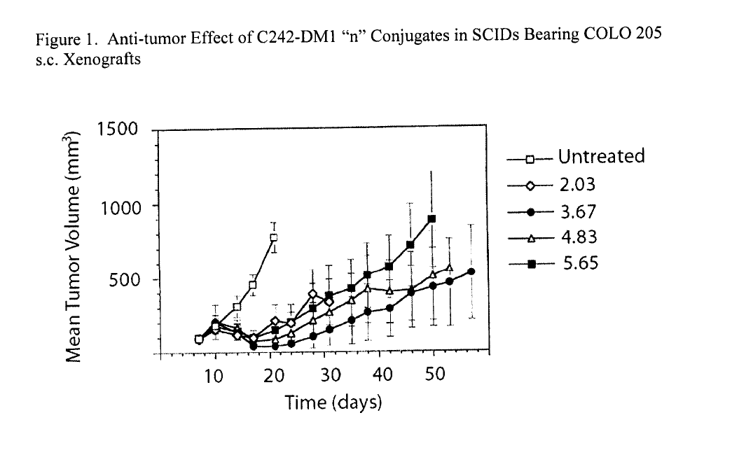

The term “average”, as used herein, is determined by spectrophotometric measurement of the absorbance of a cell binding agent (e.g., an antibody) and the drug (e.g., a maytansinoid) linked to it, as exemplified in FIG. 1.

The term “about,” as used herein in connection with numerical averages, should be understood to refer to all such numbers, including all numbers in an average and small variations therefrom.

The conjugates of the present invention comprise at least about two drugs for killing selected cell populations linked to a cell-binding agent.

In another aspect, the drug cell-binding agent conjugate is represented by the formula (D)ₙ-L-CBA, wherein D is a drug (e.g., a maytansinoid, a taxane or a CC1065 analog), n is about 2 to about 8, L is a linker, wherein the linker is selected from a cleavable linker (e.g., linkers cleavable through disulfide exchange) or a linker substantially resistant to cleavage (e.g., linkers having an N-succinimidyl ester or N-sulfosuccinimidyl ester moiety for reaction with the cell-binding agent, as well as a maleimido- or haloacetyl-based moiety) and CBA is a cell binding agent (e.g., an antibody or a fragment thereof that preferentially binds to a target cell).

In a preferred element, the drug cell binding agent conjugate (e.g., an immunoconjugate) represented by formula (May)ₙ-L-CBA, wherein May is a maytansinoid, n is about 2 to about 8, L is a linker, wherein said linker is a cleavable linker or a linker substantially resistant to cleavage; and CBA is a cell binding agent, preferably an antibody or a fragment thereof that preferentially binds to a target cell.

### Drugs

The drugs used in this invention are cytotoxic drugs capable of being linked to a cell-binding agent. Examples of suitable drugs include maytansinoids, DNA-binding drugs such as CC-1065 and its analogs, doxorubicin and its analogs, and taxoids.

### Maytansinoids

Maytansinoids that can be used in the present invention are well known in the art and can be isolated from natural sources according to known methods or prepared synthetically according to known methods.

Examples of suitable maytansinoids include maytansinol and maytansinol analogues. Examples of suitable maytansinol analogues include those having a modified aromatic ring and those having modifications at other positions.

Specific examples of suitable analogues of maytansinol having a modified aromatic ring include:

(1) C-19-dechloro (U.S. Pat. No. 4,256,746) (prepared by LAH reduction of ansamitocin P2);

(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and

(3) C-20-demethoxy, C-20-acyloxy (—OCOR), +/−dechloro (U.S. Pat. No. 4,294,757) (prepared by acylation using acyl chlorides).

Specific examples of suitable analogues of maytansinol having modifications of other positions include:

(1) C-9-SH (U.S. Pat. No. 4,424,219) (prepared by the reaction of maytansinol with H₂S or P₂S₅);

(2) C-14-alkoxymethyl (demethoxy/CH₂OR) (U.S. Pat. No. 4,331,598);

(3) C-14-hydroxymethyl or acyloxymethyl (CH₂OH or CH₂OAc) (U.S. Pat. No. 4,450,254) (prepared from Nocardia);

(4) C-15-hydroxy/acyloxy (U.S. Pat. No. 4,364,866) (prepared by the conversion of maytansinol by Streptomyces);

(5) C-15-methoxy (U.S. Pat. Nos. 4,313,946 and 4,315,929) (isolated from Trewia nudiflora);

(6) C-18-N-demethyl (U.S. Pat. Nos. 4,362,663 and 4,322,348) (prepared by the demethylation of maytansinol by Streptomyces); and

(7) 4,5-deoxy (U.S. Pat. No. 4,371,533) (prepared by the titanium trichloride/LAH reduction of maytansinol).

The synthesis of thiol-containing maytansinoids useful in the present invention is fully disclosed in U.S. Pat. Nos. 5,208,020, 5,416,064, and 7,276,497.

Maytansinoids with a thiol moiety at the C-3 position, the C-14 position, the C-15 position or the C-20 position are all expected to be useful. The C-3 position is preferred and the C-3 position of maytansinol is especially preferred. Also preferred are an N-methyl-alanine-containing C-3 thiol moiety maytansinoid, and an N-methyl-cysteine-containing C-3 thiol moiety maytansinoid, and analogues of each.

Specific examples of N-methyl-alanine-containing C-3 thiol moiety maytansinoid derivatives useful in the present invention are represented by the formulae M0, M1, M2, M3, M6 and M7.

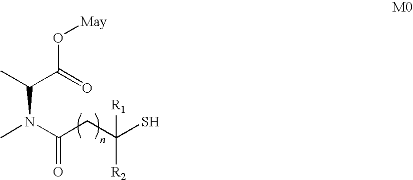

wherein the maytansinol, May, is esterified at C-3; R₁, and R₂, are independently H, linear alkyl or alkenyl having from 1 to 10 carbon atoms, branched or cyclic alkyl or alkenyl having from 3 to 10 carbon atoms, phenyl, substituted phenyl, or a heterocyclic aryl moiety, or a heterocycloalkyl moiety; and n is 1-5.

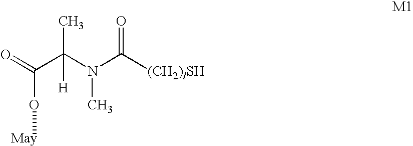

wherein:
l is an integer of from 1 to 10; and
may is a maytansinoid.

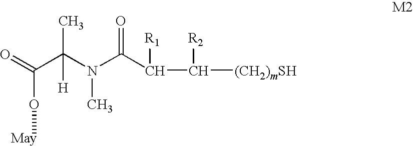

wherein:
R₁ and R₂ are H, CH₃ or CH₂CH₃, and may be the same or different;
m is 0, 1, 2 or 3; and
may is a maytansinoid.

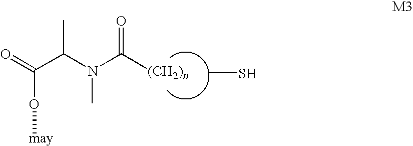

wherein:
n is an integer of from 3 to 8; and
may is a maytansinoid.

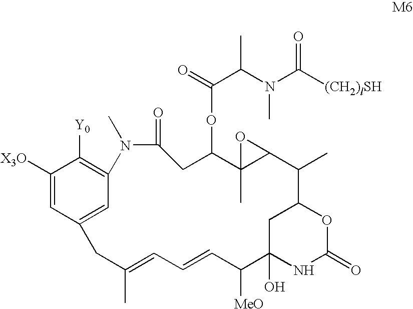

wherein:
l is 1, 2 or 3;

### Y₀ is Cl or H; and

### X₃ is H or CH₃.

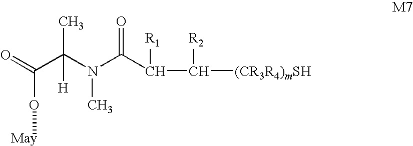

wherein:
R₁, R₂, R₃, R₄ are H, CH₃ or CH₂CH₃, and may be the same or different;
m is 0, 1, 2 or 3; and
may is a maytansinoid.

Specific examples of N-methyl-cysteine-containing C-3 thiol moiety maytansinoid derivatives useful in the present invention are represented by the formulae M4 and M5.

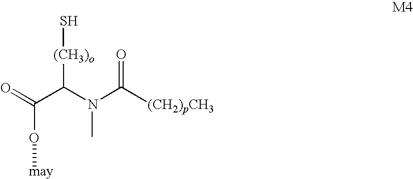

wherein:
o is 1, 2 or 3;
p is 0 or an integer of 1 to 10; and
may is a maytansinoid.

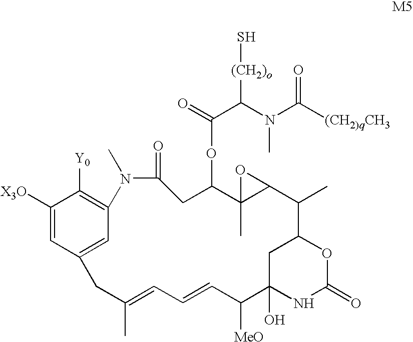

wherein:
o is 1, 2 or 3;
q is an integer of from 0 to 10;

### Y₀ is Cl or H; and

### X₃ is H or CH₃.

Preferred maytansinoids are those described in U.S. Pat. Nos. 5,208,020; 5,416,064; 6,333.410; 6,441,163; 6,716,821; RE39,151 and 7,276,497. Of these, N^{2′}-deacetyl-N^{2′}-(3-mercapto-1-oxopropyl)-maytansine (DM1) and N^{2′}-deacetyl-N^{2′}-(4-mercapto-4-methyl-1-oxopentyl) maytansine (DM4) are preferred.

Other drugs that can be used in the present invention include the following:
CC-1065 and its analogs. Preferred CC-1065 analogs are those described in U.S. Pat. Nos. 5,475,092; 5,595,499; 5,846,545; 6,534,660; 6,586,618; 6,756,397 and 7,049,316.
Doxorubicins and its analogs. Preferred doxorubicins are those described in U.S. Pat. No. 6,630,579.
Taxoids. Preferred taxoids are those described in U.S. Pat. Nos. 6,340,701; 6,372,738; 6,436,931; 6,596,757; 6,706,708; 7,008,942; 7,217,819 and 7,276,499.
Chemotherapeutic agents. “Chemotherapeutic agent” is a chemical compound useful for the treatment of cancer. Chemotherapeutic agents can be used alone for conjugation with a cell binding agent or in combination with drug-cell binding agent conjugates described herein. Preferred chemotherapeutic agents are described, for example, in U.S. Pat. No. 7,303,749.

### Analogues and Derivatives

One skilled in the art of cytotoxic drugs will readily understand that each of the drugs described herein can be modified in such a manner that the resulting compound still retains the specificity and/or activity of the starting compound. The skilled artisan will also understand that many of these compounds can be used in place of the drugs described herein. Thus, the drugs of the present invention include analogues and derivatives of the compounds described herein.

### Cell Binding Agents

The cell-binding agents used in this invention are proteins (e.g., immunoglobulin and non-immunoglobulin proteins) which bind specifically to target antigens on cancer cells. These cell-binding agents include:
antibodies including:
resurfaced antibodies (U.S. Pat. No. 5,639,641);
humanized or fully human antibodies (Humanized or fully human antibodies are selected from, but not limited to, huMy9-6, huB4, huC242, huN901, DS6, CD38, IGF-IR, CNTO 95, B-B4, trastuzumab, bivatuzumab, sibrotuzumab, and rituximab (see, e.g., U.S. Pat. Nos. 5,639,641, 5,665,357; and 7,342,110), International Patent Application WO 02/16,401, U.S. publication number 20060045877, U.S. publication number 20060127407, U.S. publication number 20050118183, Pedersen et al., (1994) J. Mol. Biol. 235, 959-973 Roguska et al., (1994) Proceedings of the National Academy of Sciences, Vol 91, 969-973, Colomer et al., Cancer Invest., 19: 49-56 (2001), Heider et al., Eur. J. Cancer, 31A: 2385-2391 (1995), Welt et al., J. Clin. Oncol., 12: 1193-1203 (1994), and Maloney et al., Blood, 90: 2188-2195 (1997)); and
fragments of antibodies such as sFv, Fab, Fab′, and F(ab′)₂ that preferentially bind to a target cell (Parham, J. Immunol. 131:2895-2902 (1983); Spring et al, J. Immunol. 113:470-478 (1974); Nisonoff et al, Arch. Biochem. Biophys. 89:230-244 (1960));

Additional cell-binding agents include other cell binding proteins and polypeptides exemplified by, but not limited to:
Ankyrin repeat proteins (DARPins; Zahnd et al., J. Biol. Chem., 281, 46, 35167-35175, (2006); Binz, H. K., Amstutz, P. & Pluckthun, A. (2005) Nature Biotechnology, 23, 1257-1268) or ankyrin-like repeats proteins or synthetic peptides described, for example, in U.S. publication number 20070238667; U.S. Pat. No. 7,101,675; WO/2007/147213; WO/2007/062466);
interferons (e.g. α, β, γ);
lymphokines such as IL-2, IL-3, IL-4, IL-6;
hormones such as insulin, TRH (thyrotropin releasing hormones), MSH (melanocyte-stimulating hormone), steroid hormones, such as androgens and estrogens; and
growth factors and colony-stimulating factors such as EGF, TGF-α, IGF-1, G-CSF, M-CSF and GM-CSF (Burgess, Immunology Today 5:155-158 (1984)).

Where the cell-binding agent is an antibody, it binds to an antigen that is a polypeptide and may be a transmembrane molecule (e.g. receptor) or a ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins, EpCAM, GD3, FLT3, PSMA, PSCA, MUC1, MUC16, STEAP, CEA, TENB2, EphA receptors, EphB receptors, folate receptor, FOLR1, mesothelin, cripto, alphaᵥbeta₆, integrins, VEGF, VEGFR, tarnsferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152 or an antibody which binds to one or more tumor-associated antigens or cell-surface receptors disclosed in US Publication No. 20080171040 or US Publication No. 20080305044 and are incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; and fragments of any of the above-listed polypeptides.

Additionally, GM-CSF, which binds to myeloid cells can be used as a cell-binding agent to diseased cells from acute myelogenous leukemia. IL-2 which binds to activated T-cells can be used for prevention of transplant graft rejection, for therapy and prevention of graft-versus-host disease, and for treatment of acute T-cell leukemia. MSH, which binds to melanocytes, can be used for the treatment of melanoma. Folic acid can be used to target the folate receptor expressed on ovarian and other tumors. Epidermal growth factor can be used to target squamous cancers such as lung and head and neck. Somatostatin can be used to target neuroblastomas and other tumor types.

Cancers of the breast and testes can be successfully targeted with estrogen (or estrogen analogues) or androgen (or androgen analogues) respectively as cell-binding agents.

Preferred antigens for antibodies encompassed by the present invention also include CD proteins such as CD2, CD3, CD4, CD5, CD6, CD8, CD11, CD18, CD19, CD20, CD22, CD26, CD30, CD33, CD37, CD38, CD40, CD44, CD56, CD79, CD105, CD138; members of the ErbB receptor family such as the EGF receptor, HER2, HER3 or HER4 receptor; cell adhesion molecules such as LFA-1, Mac1, p150.95, VLA-4, ICAM-1, VCAM, EpCAM, alpha4/beta7 integrin, and alpha v/beta3 integrin including either alpha or beta subunits thereof (e.g. anti-CD11a, anti-CD18 or anti-CD11b antibodies); growth factors such as VEGF; tissue factor (TF); TGF-β; alpha interferon (alpha-IFN); an interleukin, such as IL-8; IgE; blood group antigens Apo2, death receptor; flk2/flt3 receptor; obesity (OB) receptor; mpl receptor; CTLA-4; protein C etc., or an antibody which binds to one or more tumor-associated antigens or cell-surface receptors disclosed in US Publication No. 20080171040 or US Publication No. 20080305044 and are incorporated in their entirety by reference.

Monoclonal antibody techniques allow for the production of specific cell-binding agents in the form of monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Sensitized human cells can also be used. Another method of creating monoclonal antibodies is the use of phage libraries of sFv (single chain variable region), specifically human sFv (see, e.g., Griffiths et al, U.S. Pat. No. 5,885,793; McCafferty et al, WO 92/01047; Liming et al, WO 99/06587.)

Selection of the appropriate cell-binding agent is a matter of choice that depends upon the particular cell population that is to be targeted, but in general monoclonal antibodies and fragments thereof that preferentially bind to a target cell are preferred, if an appropriate one is available.

For example, the monoclonal antibody My9 is a murine IgG₂ₐ antibody that is specific for the CD33 antigen found on Acute Myeloid Leukemia (AML) cells (Roy et al. Blood 77:2404-2412 (1991)) and can be used to treat AML patients. Similarly, the monoclonal antibody anti-B4 is a murine IgG₁, that binds to the CD19 antigen on B cells (Nadler et al, J. Immunol. 131:244-250 (1983)) and can be used if the target cells are B cells or diseased cells that express this antigen such as in non-Hodgkin's lymphoma or chronic lymphoblastic leukemia. Similarly, the antibody N901 is a murine monoclonal IgG₁ antibody that binds to CD56 found on small cell lung carcinoma cells and on cells of other tumors of neuroendocrine origin, including merkel cell carcinoma, small cell carcinomas, neuroblastomas, glioblastomas, astrocytomas and carcinoid tumors, sarcomas, including Ewing's sarcoma, rhabdomyosarcoma and osteosarcomas, multiple myeloma, Wilm's tumor, ovarian cancers, acute myeloid leukemia and NK/T-cell malignancies (lymphomas and leukemias) (Roy et al. J. Nat. Cancer Inst. 88:1136-1145 (1996)), huC242 antibody that binds to the CanAg antigen, Trastuzumab that binds to HER2/neu, and anti-EGF receptor antibody that binds to EGF receptor.

The cell binding agent can be conjugated to the cytotoxic drugs by methods previously described (U.S. Pat. Nos. 6,013,748; 6,441,1631, 6,716,821, US Application 20050169933; WO2006/034488 A2).

### Linkers

The conjugates may be prepared by in vitro methods. In order to link a drug to the cell-binding agent, a linking group is used. Suitable linking groups are well known in the art and include disulfide groups, acid labile groups, photolabile groups, peptidase labile groups, and esterase labile groups. Preferred linking groups are disulfide groups. For example, conjugates can be constructed using a disulfide exchange reaction between the cell-binding agent and the drug. The drugs also can be linked to a cell-binding agent through an intermediary carrier molecule such as serum albumin.

Thus, in accordance with the invention, the cell-binding agent is modified by reacting a bifunctional crosslinking reagent with the cell-binding agent, thereby resulting in the covalent attachment of a linker molecule to the cell-binding agent. As used herein, a “bifunctional crosslinking reagent” is any chemical moiety that covalently links a cell-binding agent to a drug, such as the drugs described herein. In a preferred aspect of the invention, a portion of the linking moiety is provided by the drug. In this respect, the drug comprises a linking moiety that is part of a larger linker molecule that is used to join the cell-binding agent to the drug. For example, to form the maytansinoid, DM1, the side chain at the C-3 hydroxyl group of maytansine is modified to have a free sulfhydryl group (SH). This thiolated form of maytansine can react with a modified cell-binding agent to form a conjugate. Therefore, the final linker is assembled from two components, one of which is provided by the crosslinking reagent, while the other is provided by the side chain from DM1.

Any suitable bifunctional crosslinking reagent can be used in connection with the invention, so long as the linker reagent provides for retention of the therapeutic, e.g., cytotoxic, and targeting characteristics of the drug and the cell-binding agent, respectively. Preferably, the linker molecule joins the drug to the cell-binding agent through chemical bonds (as described above), such that the drug and the cell-binding agent are chemically coupled (e.g., covalently bonded) to each other. Preferably, the linking reagent is a cleavable linker. More preferably, the linker is cleaved under mild conditions, i.e., conditions within a cell under which the activity of the drug is not affected. Examples of suitable cleavable linkers include disulfide linkers, acid labile linkers, photolabile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Photo labile linkers are useful at the body surface and in many body cavities that are accessible to light. Furthermore, infrared light can penetrate tissue. Peptidase labile linkers can be used to cleave certain peptides inside or outside cells (see e.g., Trouet et al., Proc. Natl. Acad. Sci. USA, 79: 626-629 (1982), and Umemoto et al., Int. J. Cancer, 43: 677-684 (1989)).

Preferably the drug is linked to a cell-binding agent through a disulfide bond. The linker molecule comprises a reactive chemical group that can react with the cell-binding agent. Preferred reactive chemical groups for reaction with the cell-binding agent are N-succinimidyl esters and N-sulfosuccinimidyl esters. Additionally the linker molecule comprises a reactive chemical group, preferably a dithiopyridyl group, that can react with the drug to form a disulfide bond. Particularly preferred linker molecules include, for example, N-succinimidyl 3-(2-pyridyldithio)propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), and other reactive cross-linkers, such as those described in U.S. Pat. No. 6,913,748, which is incorporated herein in its entirety by reference. Even more preferred linker molecule is N-succinimidyl 4-(2-pyridyldithio)pentanoate (SPP).

While cleavable linkers preferably are used in the inventive method, a non-cleavable linker also can be used to generate the above-described conjugate. A non-cleavable linker is any chemical moiety that is capable of linking a drug, such as a maytansinoid, a Vinca alkaloid, a dolastatin, an auristatin, or a cryptophycin, to a cell-binding agent in a stable, covalent manner. Thus, non-cleavable linkers are substantially resistant to acid-induced cleavage, light-induced cleavage, peptidase-induced cleavage, esterase-induced cleavage, and disulfide bond cleavage, at conditions under which the drug or the cell-binding agent remains active.

Suitable crosslinking reagents that form non-cleavable linkers between a drug and the cell-binding agent are well known in the art. Examples of non-cleavable linkers include linkers having an N-succinimidyl ester or N-sulfosuccinimidyl ester moiety for reaction with the cell-binding agent, as well as a maleimido- or haloacetyl-based moiety for reaction with the drug. Crosslinking reagents comprising a maleimido-based moiety include N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP).

Other crosslinking reagents lacking a sulfur atom that form non-cleavable linkers can also be used in the inventive method. Such linkers can be derived from dicarboxylic acid based moieties. Suitable dicarboxylic acid based moieties include, but are not limited to, α,ω-dicarboxylic acids of the general formula shown below:

HOOC—Xₗ—Yₙ—Zₘ—COON

wherein X is a linear or branched alkyl, alkenyl, or alkynyl group having 2 to 20 carbon atoms, Y is a cycloalkyl or cycloalkenyl group bearing 3 to 10 carbon atoms, Z is a substituted or unsubstituted aromatic group bearing 6 to 10 carbon atoms, or a substituted or unsubstituted heterocyclic group wherein the hetero atom is selected from N, O or S, and wherein l, m, and n are each 0 or 1, provided that l, m, and n are all not zero at the same time.

Many of the non-cleavable linkers disclosed herein are described in detail in co-pending provisional patent applications 61/049, 291; 61/147, 966 and 61/049, 289 and in U.S. Patent publication number 20050169933, each of which is expressly incorporated herein by reference.

Alternatively, as disclosed in U.S. Pat. No. 6,441,163 B1, the drug can be first modified to introduce a reactive ester suitable to react with a cell-binding agent. Reaction of these maytansinoids containing an activated linker moiety with a cell-binding agent provides another method of producing a cleavable or non-cleavable cell-binding agent maytansinoid conjugate.

### Therapeutic Use

The conjugates of the present invention can be administered in vitro, in vivo and/or ex vivo to treat patients and/or to modulate the growth of selected cell populations including, for example, cancer of the lung, blood, plasma, breast, colon, prostate, kidney, pancreas, brain, bones, ovary, testes, and lymphatic organs; autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like. Preferably, the conjugates and chemotherapeutic agents of the invention are administered in vitro, in vivo and/or ex vivo to treat cancer in a patient and/or to modulate the growth of cancer cells, including, for example, cancer of the blood, plasma, lung, breast, colon, prostate, kidney, pancreas, brain, bones, ovary, testes, and lymphatic organs; more preferably lung, colon prostrate, plasma, blood or colon cancer. In a most preferred embodiment, the cancer is multiple myeloma.

“Modulating the growth of selected cell populations” includes inhibiting the proliferation of selected cell populations (e.g., multiple myeloma cell populations, such as MOLP-8 cells, OPM2 cells, H929 cells, and the like) to produce more cells; reducing the rate of increase in cell division as compared, for example, to untreated cells; killing selected cell populations; and/or preventing selected cell populations (such as cancer cells) from metastasizing. The growth of selected cell populations can be modulated in vitro, in vivo or ex vivo.

In the methods of the present invention, the conjugates and chemotherapeutic agents can be administered in vitro, in vivo, or ex vivo separately or as components of the same composition. The combined administration includes co-administration, using separate formulations or a single pharmaceutical formulation, and consecutive administration in either order, wherein preferably there is a time period while both (or all) active agents simultaneously exert their biological activities.

The conjugates and chemotherapeutic agents can be used with suitable pharmaceutically acceptable carriers, diluents, and/or excipients, which are well known, and can be determined by one of skill in the art as the clinical situation warrants. Examples of suitable carriers, diluents and/or excipients include: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

The compounds and compositions described herein may be administered in appropriate form, preferably parenterally, more preferably intravenously. For parenteral administration, the compounds or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants.

The compositions can also be in the form of sterile solid compositions that can be dissolved or dispersed in sterile water or any other injectable sterile medium.

The “therapeutically effective amount” of the chemotherapeutic agents and conjugates described herein refers to the dosage regimen for modulating the growth of selected cell populations and/or treating a patient's disease, and is selected in accordance with a variety of factors, including the age, weight, sex, diet and medical condition of the patient, the severity of the disease, the route of administration, and pharmacological considerations, such as the activity, efficacy, pharmacokinetic and toxicology profiles of the particular compound used. The “therapeutically effective amount” can also be determined by reference to standard medical texts, such as the Physicians Desk Reference 2004. The patient is preferably an animal, more preferably a mammal, most preferably a human. The patient can be male or female, and can be an infant, child or adult.

Examples of suitable protocols of conjugate administration are as follows. Conjugates can be given daily for about 5 days either as an i.v. bolus each day for about 5 days, or as a continuous infusion for about 5 days.

Alternatively, the conjugates can be administered once a week for six weeks or longer. As another alternative, the conjugates can be administered once every two or three weeks. Bolus doses are given in about 50 to about 400 ml of normal saline to which about 5 to about 10 ml of human serum albumin can be added. Continuous infusions are given in about 250 to about 500 ml of normal saline, to which about 25 to about 50 ml of human serum albumin can be added, per 24 hour period. Dosages will be about 10 pg to about 1000 mg/kg per person, i.v. (average of about 100 ng to about 100 mg/kg; more preferably in the average of about 100 ng to about 10 mg/kg).

About one to about four weeks after treatment, the patient can receive a second course of treatment. Specific clinical protocols with regard to route of administration, excipients, diluents, dosages, and times can be determined by the skilled artisan as the clinical situation warrants.

The present invention also provides pharmaceutical kits comprising one or more containers filled with one or more of the ingredients of the pharmaceutical compounds and/or compositions of the present invention, including, one or more conjugates and one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products.

Cancer therapies and their dosages, routes of administration and recommended usage are known in the art and have been described in such literature as the Physician's Desk Reference (PDR). The PDR discloses dosages of the agents that have been used in treatment of various cancers. The dosing regimen and dosages of these aforementioned chemotherapeutic agents and conjugates that are therapeutically effective will depend on the particular cancer being treated, the extent of the disease and other factors familiar to the physician of skill in the art and can be determined by the physician. For example, the 2006 edition of the Physician's Desk Reference discloses that Taxotere (see p. 2947) is an inhibitor of tubulin depolymerization; Doxorubicin (see p 786), Doxil (see p 3302) and oxaliplatin (see p 2908) are DNA interacting agents, Irinotecal (see p. 2602) is a Topoisomerase I inhibitor, Erbitux (see p 937) and Tarceva (see p 2470) interact with the epidermal growth factor receptor. The contents of the PDR are expressly incorporated herein in their entirety by reference. One of skill in the art can review the PDR, using one or more of the following parameters, to determine dosing regimens and dosages of the chemotherapeutic agents and conjugates, which can be used in accordance with the teachings of this invention. These parameters include:

1. Comprehensive index

### a) by Manufacturer

b) Products (by company's or trademarked drug name)
c) Category index (for example, “antihistamines”, “DNA alkylating agents” taxanes etc.)
d) Generic/chemical index (non-trademark common drug names)
2. Color images of medications
3. Product information, consistent with FDA labeling
a) Chemical information

### b) Function/action

### c) Indications & Contraindications

d) Trial research, side effects, warnings

The entire contents of each of the foregoing references, patent applications, and patents are expressly incorporated by reference in their entirety including, without limitation, the specification, claims, and abstract, as well as any figures, tables, or drawings thereof.

### EXAMPLES

The invention will now be described by reference to non-limiting examples. Unless otherwise specified, all percents and ratios are by volume.

### Example 1

### Preparation of C242-DM1 Conjugates Bearing Different Maytansinoid Loads

### Materials and Methods:

Forty-four female CB.17 SCID mice (Dept. of Radiation Oncology, Massachusetts General Hospital, Boston, Mass.) were inoculated with 2×10⁶ Colo 205 (ATCC CRL 222) cells in 0.1 mL RPMI under the skin of their right flank. When tumors reached 50-100 mm³, seven days later, mice were identified using Sharpie markers, and randomized based upon tumor size into five groups (n=7/group). Groups of mice were then treated as follows:

1. Untreated control

2. 75 μg/kg/day C242-DM1_{2.03} (1304−196)×5 qd, I.V.

3. 75 μg/kg/day C242-DM1_{3.67} (1304−184)×5 qd, I.V.

4. 75 μg/kg/day C242-D.M1_{4.83} (1304−195)×5 qd, I.V.

5. 75 μg/kg/day C242-DM1_{5.65} (1304−172)×5 qd, I.V.

All dose formulations were made on each day of dosing using sterile phosphate buffered saline (PBS) as the diluent. Mice were dosed based upon their individual day 7 weight by varying the volume of injection. Tumor volume was calculated using the formula a×b²/0.5, where a and b are the long and short lengths of the tumor, respectively. Tumors were measured using calipers at least two times per week until the mean tumor volume of each group reached a size ≧1000 mm³, or tumors became ulcerated and burst.

### Results:

FIG. 1 illustrates the anti-tumor effect of C242-DM1 containing 2.03, 3.67, 4.83, or 5.65 DM1 molecules/C242 molecule. C242-DM1 containing 2.03, 3.67, 4.83 and 5.65 DM1/C242 delayed the growth of Colo 205 tumors 5, 15, 11 and 5 days, respectively. There appeared to be a significant difference in tumor growth delay of mice treated with C242-DM1 containing 3.67 DM1/C242 when compared to C242-DMI containing 2.03 and 5.65 DM1/C242, the latter two conjugates being the least efficacious.

### Example 2

### Preparation of HUN901-DM1 Conjugates Bearing Different Maytansinoid Loads

In the first step, the huN901 antibody was reacted with the modifying agent 4-[2-pyridyldithio]pentanoic acid-N-hydroxysuccinimde ester (SPP) to introduce dithiopyridyl groups. A solution of huN901 antibody was diluted to 8 mg/mL in an aqueous buffer containing 0.05 M potassium phosphate, 0.05 M sodium chloride and 2 mM ethylenediaminetetra-acetic acid (EDTA), pH 6.5 and was reacted with SPP molar excesses ranging from 2 equivalents to 15 equivalents in dimethylacetamide (DMA), such that the reaction mixture was 5% DMA total. The reaction mixture was stirred at room temperature for 90 min and purified over a Sephadex G25 pre-packed column (NAP25) that had been previously equilibrated into the aforementioned buffer. The molar ratio of released SPy per mole of antibody was calculated by measuring the A₂₈₀ of the sample and then the increase in the A₃₄₃ of the sample after adding dithiothreitol (DTT) (0.05 mL of 1 M DTT per mL of sample). The concentration of DTT-released SPy was calculated using an extinction coefficient ε_{343 nm} of 8080 M⁻¹cm⁻¹. The concentration of antibody was calculated using an ε_{280 nm} of 195,440 M⁻¹cm⁻¹ after subtracting the contribution of SPy absorbance at 280 nm (A_{343 nm} post DTT×5180/8080) from the total A_{280 nm} measured before DTT addition. The molar ratio of SPy:Ab was then calculated. The mg/mL (g/L) concentration of Ab was calculated using a molecular weight of 146,400 g/mol. The average number of molecules linked is shown in the table below.

The modified antibody was diluted to 2.5 mg/mL in the above buffer at pH 6.5 and then treated with a solution of DM1 in DMA, with a drug excess of 1.7 drugs per linker, such that the final concentration of DMA in the buffer was 5%. The conjugation mixture was reacted at room temperature overnight. The reaction mixture was purified by passage through Sephadex G25 pre-packed column (NAP25), that had been previously equilibrated in 10 mM citrate, 135 mM NaCl, pH 5.5. The purified conjugate was assayed spectrophotometrically to determine the drug to antibody ratio. The Ab and DM1 both absorb at the two wavelengths used to measure each component separately, i.e., 280 and 252 nm. The extinction coefficient at 280 nm for the huN901 antibody is 195,440 M⁻¹cm⁻¹ and for DM1 is 5700 M⁻¹ cm⁻¹. The 252 nm/280 nm absorbance ratio of the antibody is 0.352 and 4.7 for the DM1. The components were quantified using the following algebraic expressions which account for the contribution of each component at each wavelength (C_{Ab} is the molar concentration of the huHUN901 antibody and C_{D} is the molar concentration of DM1):

$${C}_{D} = \frac{{A}_{252} - 0.352 {A}_{280}}{24783.6}$$

$${C}_{Ab} = \frac{{A}_{280} - 5700 {C}_{D}}{195 , 440}$$

The ratio of DM1:Ab was then calculated. The mg/mL (g/L) concentration of antibody was calculated using a molecular weight of 146,400 g/mole and the concentration of DM1 is calculated using a molecular weight of 737.3 g/mole (linked DM1). The average number of drugs incorporated is summarized in the table below.

| SPP | SPy | |
|---|---|---|
| excess | incorporation | # DM1/Ab |
| 2 | 1.6 | 1.0 |
| 4 | 2.9 | 2.0 |
| 6 | 4.2 | 3.0 |
| 8 | 5.8 | 4.0 |
| 12 | 7.9 | 5.8 |
| 15 | 10.3 | 6.6 |

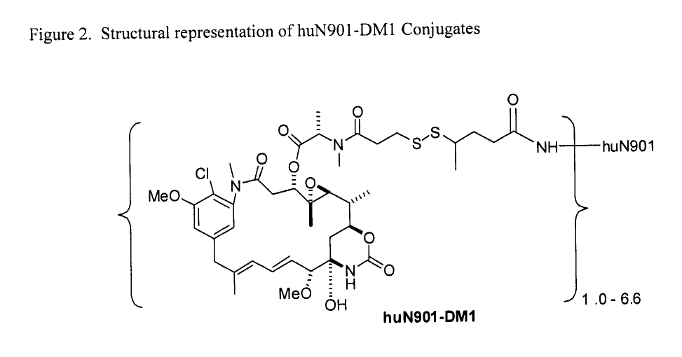

A structural representation of huN90′-SPP-DM1 is shown in FIG. 2.

### Example 3

### Binding Studies

Binding of the huN901 antibody and its DM1 conjugates was evaluated on whole cells by an indirect immunofluorescence assay utilizing flow cytometry. Cells (5×10⁴ per well) were plated in a round bottom 96-well plate (Falcon #2072) and incubated at 4° C. for 3 hours with serial dilutions of huN901 antibody or huN901-SPP-DM1 in 0.2 mL of alpha-MEM medium (Invitrogen, Inc.) supplemented with 2% (v/v) normal goat serum (Sigma). Each sample was run in duplicate. Control wells contained cells and the medium, but lacked the antibody or the conjugates. The cells were then washed with 0.2 mL cold (4° C.) medium and stained with fluorescein-labeled goat anti-human IgG antibody at 4° C. for 50 min. The cells were again washed with the medium, fixed in 1% formaldehyde/PBS solution and then analyzed using a FACSCalibur flow cytometer (BD Biosciences, San Jose, Calif.). Apparent Kd values were determined by curve fitting analysis using Prism software.

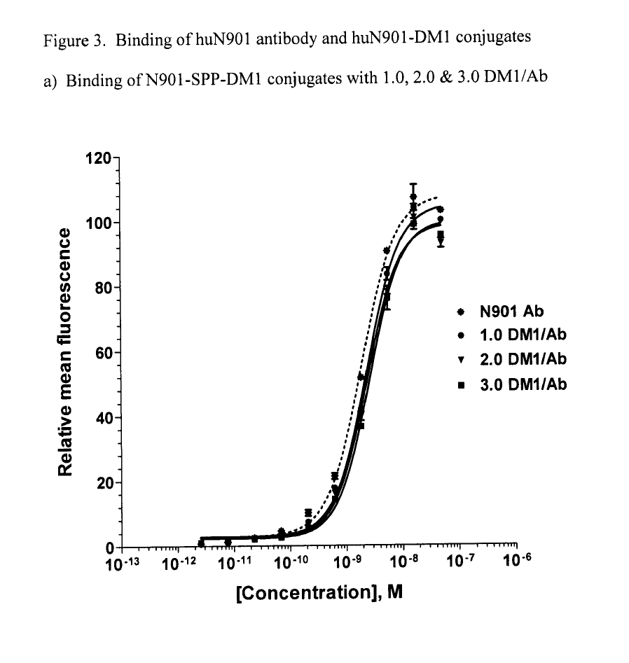

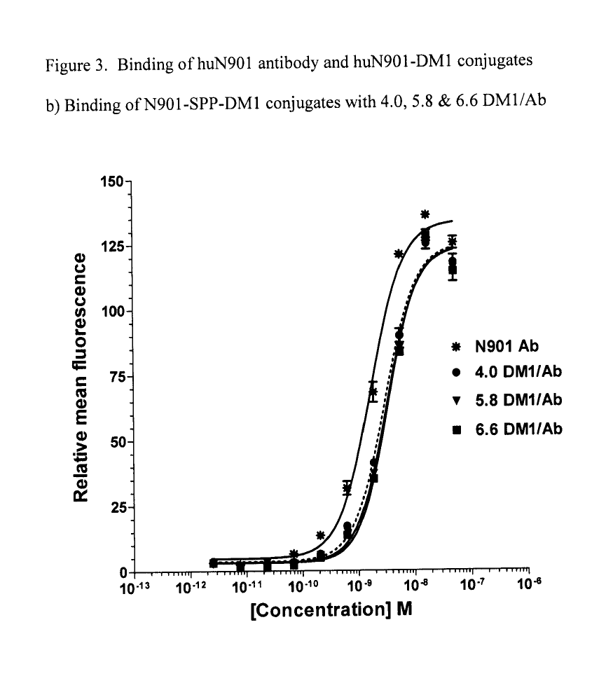

The results are shown in FIGS. 3a, b. Conjugates bearing 1, 2 or 3 DM1/antibody bind with similar affinity as the unconjugated huN901 antibody (FIG. 3a), suggesting that conjugation did not affect binding. Conjugates bearing 4, 5.8 or 6.6 DM1/antibody show slightly diminished binding compared to the unconjugated antibody (FIG. 3b).

### Example 4

### In Vitro Cytotoxicity Assays

The cytotoxicity study was performed using a clonogenic assay. CD56-expressing CHD163 cells were plated into 6-well culture dishes at a constant number of 3,000 cells per well. The cells were incubated with varying concentrations of huN901-DM1 (with DM1 load ranging from 1.0 to 6.6 DM1 molecules linked per antibody) in Dulbecco's modified Eagles Medium (DMEM, Biowhittaker, Walkersville, Md.) with L-glutamine supplemented with 10% fetal bovine serum (Hyclone, Logan, Utah) and 50 μg/ml of gentamycin sulfate (Life Technologies, Rockville, Md.) for 7 days. The cultures were then fixed and stained with 0.2% crystal violet in 10% formalin/PBS, and the colonies were counted. The surviving fraction of cells exposed to the conjugates was determined by dividing the number of colonies in wells that were exposed to the conjugate by the number of colonies in the control non-treated wells.

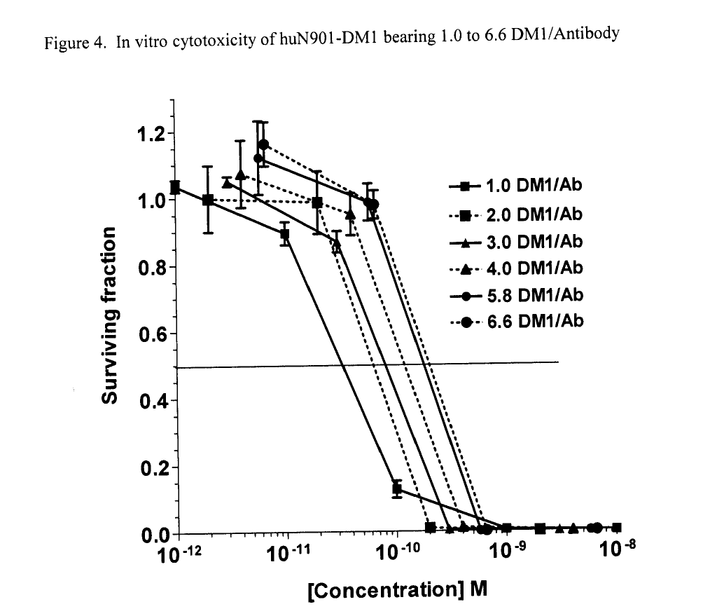

The results are shown in FIG. 4. All conjugates were potent, with IC₅₀ values ranging from 2×10⁻¹¹ M to 1×10⁻¹⁰ M, with the conjugate bearing just 1 DM1/Ab being surprisingly the most potent, with an IC₅₀ value of 2×10⁻¹¹ M. Conjugates with DM1 load between 2-4 DM1/Ab, were also surprisingly more potent than that bearing 5.8 DM1/Ab.

### Example 5

### In Vivo Efficacy Studies

The in vivo anti-tumor activity of huN901-SPP-DM1 conjugates with a range of maytansinoid-to-antibody ratios (DM1/Ab values) was evaluated in female CB.17 SCID mice bearing OPM2 human multiple myeloma tumors. The OPM2 human multiple myeloma cell line was maintained in RPMI-1640 medium containing 2 mM L-glutamine, supplemented with 10% heat-inactivated fetal bovine serum and 50 μg/ml gentamicin sulfate in a 37° C., 6% CO²-humidified chamber.

CB.17 SCID mice (96 female animals, five weeks old) were obtained from Charles River Laboratories. Upon receipt, the animals were observed for 9 days prior to study initiation. Animals showed no signs of disease or illness upon arrival, or prior to administration of the test materials. Mice were inoculated with 1×10⁷ OPM2 cells in 0.1 mL serum free medium by subcutaneous injection in the right flank area.

Groups and Treatments: (8 mice/group)

1. PBS Control
Groups 2-7: huN901-SPP-DM1-10 mg/kg conjugate, single injection

2. 1.0 DM1/Ab

3. 2.0 DM1/Ab

4. 3.0 DM1/Ab

5. 4.0 DM1/Ab

6. 5.8 DM1/Ab

7. 6.6 DM1/Ab

Mice were treated intravenously 20 days post cell inoculation. The randomized tumor volumes ranged from 98.8 to 185.5 (136.5±20.9, Mean±SD) mm³. Body weights of the mice ranged from 15.7 to 21.4 (19.4±1.2, Mean±SD) grams. Mice in the treatment groups were dosed with 10 mg/kg conjugate based on individual body weight.

Tumor size was measured twice to three times weekly in three dimensions using a caliper. The tumor volume was expressed in mm³ using the formula V=Length×Width×Height×½. A mouse was considered to have a partial regression (PR) when tumor volume was reduced by 50% or greater, complete tumor regression (CR) when no palpable tumor could be detected. Body weight of all the mice was measured twice per week as a rough index of drug toxicity. Animals were sacrificed when the tumor volume was larger than 1500 mm³ or necrotic, or if body weight dropped by 20% more at any point in the study.

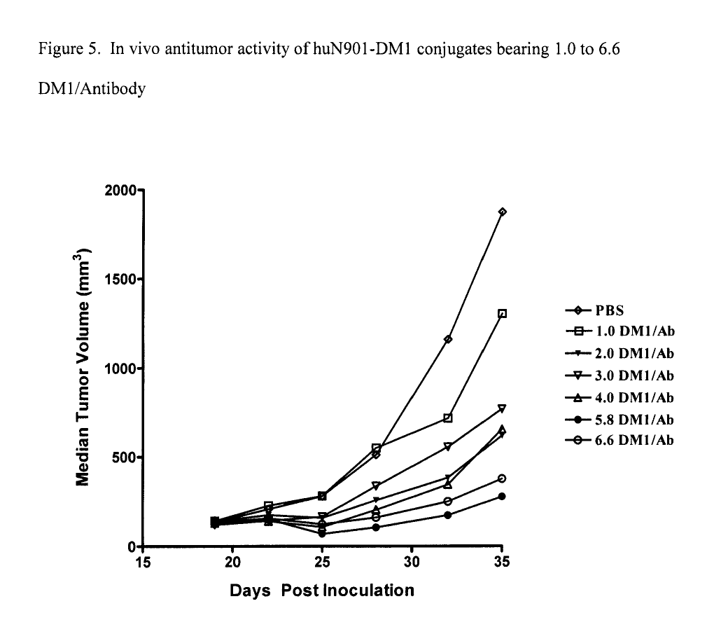

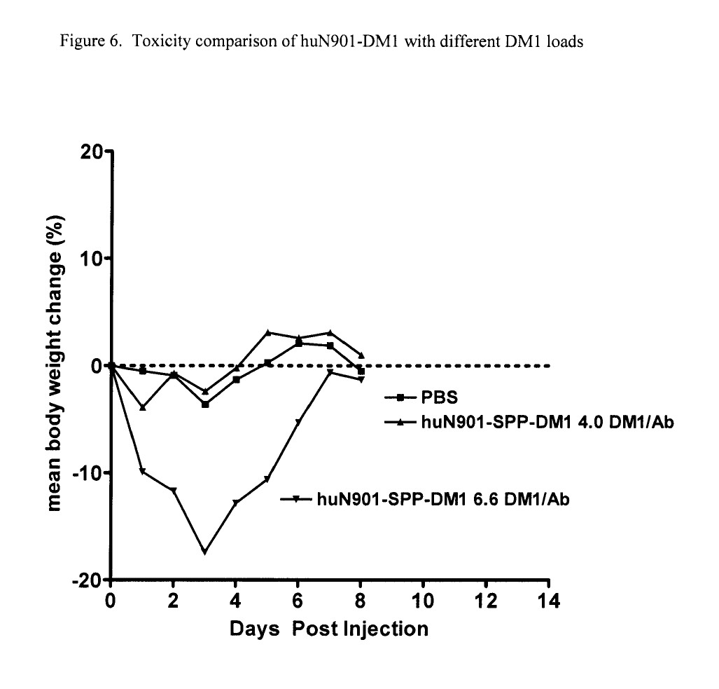

The results are shown in FIG. 5. huN901-DM1 with 1.0 DM1/Ab was inactive with no response noted. At the 2.0 DM1/Ab, huN901-SPP-DM1 was active, with two partial tumor regressions. There were one partial and one complete regression in the 3.0 DM1/Ab and the 4.0 DM1/Ab groups. The 4 DM1/Ab group was considered active with a T/C=32%. The 5.8 DM1/Ab of huN901-SPP-DM1 showed the highest activity with a T/C value of 16% and five partial regressions and two complete regressions. Surprisingly, the 6.6 DM1/Ab group of huN901-SPP-DM1, had a higher T/C value of 23% compared to the 5.8 DM1/Ab group, suggesting lower antitumor activity. The T/C value is a measure of antitumor activity, with a lower T/C value signifying greater anti-tumor activity (see Bissery, M. et al. Experimental Antitumor Activity of Taxotere (RP 56976, NSC 628503), a Taxol Analogue. Cancer Res. 51, 4845-4852, September 1991).

Thus, the data shows that conjugates with low maytansinoid load (<2 DM1/Antibody) are less active. This finding was unexpected considering that the most potent huN901-DM1 conjugate in vitro had just 1 DM1/Ab. In addition, unexpectedly, increasing the maytansinoid load above 5.8 DM1/Antibody does not provide improvement in antitumor activity. Increasing the maytansinoid load above 5.8 will increase the cost of goods without any therapeutic benefit. Also, increasing the maytansinoid load beyond 5.8 is likely to result in increased systemic toxicity without any increase in therapeutic benefit.

### Example 6

### Tolerability Studies

The toxicity of two different drug loads of huN901-DM1 (4.0 DM1/Ab versus 6.6 DM1/Ab) was compared in mice. Mice (seven weeks old, CD-1, Charles River laboratories) were randomly divided into three groups of four mice each. Body weights ranged from 23.7 to 26.3 (24.9±1.0, Mean±SD) grams. Mice were dosed with either a) PBS or b) huN901-DM1 at 4.0 DM1/antibody or c) huN901-DM1 at 6.6 DM1/antibody load based on individual body weight. Administration was carried out intravenously with a 1.0 ml syringe fitted with a 27 gauge, ½ inch needle. Mice were weighed and observed daily for signs of toxicity or morbidity. Any mouse exhibiting greater than 20% body weight loss was sacrificed.

The results are shown in FIG. 6. Mice in PBS and huN901-DM1 4.0 DM1/Ab treatment groups did not lose body weight or exhibit any signs of distress or morbidity over the study period (8 days post injection). Severe body weight loss was observed in the huN901-DM1 6.6 DM1/Ab treatment group beginning on day 1 post injection, with body weight nadir occurring on day 3 post injection. One mouse was sacrificed due to body weight loss greater than 20% of initial body weight. Thus, despite the efficacy observed, conjugates with high DM1/Ab of 6.6 drugs per antibody are more toxic and the therapeutic index at DM1/Ab of 6.6 is too low to be useful.

1. A drug cell binding agent conjugate of the formula:
(D)ₙ-L-CBA,
wherein, D is a drug; n is about 2 to about 8, L is a linker; and, CBA is a cell binding agent.

2. The conjugate of claim 1, wherein n is about 2.5 to about 6.6.

3. The conjugate of claim 1, wherein n is about 2.5 to about 5.8.

4. The conjugate of claim 1, wherein n is about 2.5 to about 4.5.

5. The conjugate of claim 1, wherein said drug is selected from a maytansinoid, a CC1065 analog, a taxane, a doxorubicin and a chemotherapeutic agent.

6. The conjugate of claim 1, wherein said maytansinoid is a compound of formula: 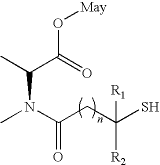
wherein the maytansinol, May, is esterified at C-3; R₁, and R₂, are independently H, linear alkyl or alkenyl having from 1 to 10 carbon atoms, branched or cyclic alkyl or alkenyl having from 3 to 10 carbon atoms, phenyl, substituted phenyl, or a heterocyclic aryl moiety, or a heterocycloalkyl moiety; and n is 1-5.

7. The conjugate of any one of claims 2 to 6, wherein said maytansinoid is N^{2′}-deacetyl-N^{2′}-(3-mercapto-1-oxopropyl)-maytansine (DM1) or N^{2′}-deacetyl-N^{2′}-(4-mercapto-4-methyl-1-oxopentyl) maytansine (DM4).

8. The conjugate of claim 1, wherein said linker is selected from a non-cleavable linker or a cleavable linker.

9. The conjugate of claim 8, wherein said non-cleavable linker is substantially resistant to acid-induced cleavage, photolabile-induced cleavage, peptidase-induced cleavage, esterase-induced cleavage, cleavage of disulfide link through thiol of N-methyl cysteine or N-methyl-homocysteine.

10. The conjugate of claim 8, wherein said cleavable linker is a acid-labile linker, a photolabile linker, a peptidase-labile linker, an esterase labile linker, a disulfide linked through thiol of N-methyl cysteine or N-methyl-homocysteine.

11. The conjugate of claim 8, wherein said cleavable linker is a disulfide linked through thiol of N-methyl cysteine or N-methyl-homocysteine.

12. The conjugates of claim 8, wherein said cleavable linker is N-succinimidyl 4-(2-pyridyldithio)pentanoate (SPP).

13. The conjugate of claim 1, wherein said cell-binding agent is an antibody, a single chain antibody, an antibody fragment that preferentially binds to a target, a monoclonal antibody, a single chain monoclonal antibody, a fragment of a monoclonal antibody that preferentially binds to a target cell, a bispecific antibody fragment that preferentially binds to a target cell, a lymphokine, a cytokine, a hormone, a growth factor, an enzyme, or a nutrient-transport molecule.

14. The conjugate of claim 1, wherein said cell-binding agent is a resurfaced monoclonal antibody, a resurfaced single chain monoclonal antibody, or a resurfaced monoclonal antibody fragment that preferentially binds to a target cell.

15. The conjugate of claim 1, wherein said cell-binding agent is a human or a humanized monoclonal antibody, a human or humanized single chain monoclonal antibody, or a human or humanized monoclonal antibody fragment that preferentially binds to a target cell.

16. The conjugate of claim 13, wherein said antibody is a chimeric antibody, a chimeric antibody fragment that preferentially binds to a target cell, a domain antibody, or a domain antibody fragment thereof that preferentially binds to a target cell.

17. The conjugate of claim 13, wherein said antibody is My9-6, B4, C242, N901, DS6, EphA2 receptor, CD38, IGF-IR, CNTO 95, B-B4, trastuzumab, bivatuzumab, sibrotuzumab, or rituximab.

18. The conjugate of claim 13, wherein said antibody is humanized or resurfaced My9-6, B4, C242, N901, DS6, CD38, IGF-IR, B-B4, trastuzumab, bivatuzumab, sibrotuzumab, or rituximab.

19. The conjugate of claim 13, wherein said antibody is humanized or resurfaced N901.

20. The conjugate of claim 1, wherein said cell-binding agent binds to target cells selected from tumor cells; virus infected cells, microorganism infected cells, parasite infected cells, autoimmune cells, activated cells, myeloid cells, activated T-cells, B cells, or melanocytes; cells expressing one or more of IGF-IR, CanAg, EGFR, EphA2 receptor, MUC1, MUC16, VEGF, TF, MY9, anti-B4, EpCAM, CD2, CD3, CD4, CD5, CD6, CD11, CD 11a, CD18, CD19, CD20, CD22, CD26, CD30, CD33, CD37, CD38, CD40, CD44, CD56, CD79, CD105, CD138, EphA receptors, EphB receptors, EGFr, EGFRvIII, HER2/neu, HER3, mesothelin, cripto, alphaᵥbeta₃ integrin, alphaᵥbeta₅ integrin, alphaᵥbeta₆ integrin, Apo2, and C242 antigens; or cells expressing insulin growth factor receptor, epidermal growth factor receptor, and folate receptor.

21. The conjugate of claim 20, wherein the tumor cells are selected from breast cancer cells, prostate cancer cells, ovarian cancer cells, colorectal cancer cells, gastric cancer cells, squamous cancer cells, small-cell lung carcinoma cells, testicular cancer cells, multiple myeloma cells, and cells from NK/T-cell malignancies.

22. The conjugate of claim 20, wherein the tumor cells are selected from tumor cells of neuroendocrine origin that express CD56.

23. The conjugate of claim 20, wherein the tumor cells of neuroendocrine origin that express CD56 are cells from merkel cell carcinoma.

24. The conjugate of claim 1, wherein D is N^{2′}-deacetyl-N^{2′}-(3-mercapto-1-oxopropyl)-maytansine (DM1), n is about 2.5 to about 6.6, L is N-succinimidyl 4-(2-pyridyldithio)pentanoate (SPP), and CBA is a resurfaced or humanized antibody, a resurfaced or humanized single chain antibody or a resurfaced or humanized antibody fragment that binds to cells expressing the CD56 antigen.

25. The conjugate of claim 24, wherein n is about 2.5 to about 5.8.

26. The conjugate of claim 25, wherein n is about 2.5 to about 4.5.

27. The conjugate of any one of claims 24 to 26, wherein CBA is a resurfaced or humanized antibody that binds to the same epitope as N901, a resurfaced or humanized single chain antibody that binds to the same epitope as N901, or a resurfaced or humanized antibody fragment that binds to the same epitope as N901.

28. The conjugate of any one of claims 24 to 26, wherein CBA is a resurfaced or humanized antibody N901, a resurfaced or humanized single chain antibody that binds to antibody N901, or a resurfaced or humanized antibody binding fragment or antibody N901.

29. The conjugate of any one of claims 24 to 26, wherein CBA is huN901, single chain huN901, or an antigen binding fragment of huN901.

30. The conjugate of claim 27, wherein the CBA binds to small-cell lung carcinoma cells that express the CD56 antigen.

31. The conjugate of claim 28, wherein the CBA binds to small-cell lung carcinoma cells that express the CD56 antigen.

32. The conjugate of claim 29, wherein the CBA binds to small-cell lung carcinoma cells that express the CD56 antigen.

33. A pharmaceutical composition comprising an effective amount of the drug-cell-binding agent conjugate of any one of claims 1, 24, 25 and 26, a pharmaceutically acceptable salt or solvate thereof, and a pharmaceutically acceptable carrier, diluent or excipient.

34. A pharmaceutical composition comprising an effective amount of the drug-cell-binding agent conjugate of claim 27, a pharmaceutically acceptable salt or solvate thereof, and a pharmaceutically acceptable carrier, diluent or excipient.

35. A pharmaceutical composition comprising an effective amount of the drug-cell-binding agent conjugate of claim 28, a pharmaceutically acceptable salt or solvate thereof, and a pharmaceutically acceptable carrier, diluent or excipient.

36. A pharmaceutical composition comprising an effective amount of the drug-cell-binding agent conjugate of claim 29, a pharmaceutically acceptable salt or solvate thereof, and a pharmaceutically acceptable carrier, diluent or excipient.

37. A method for treating tumor sensitive to treatment with said method, said method comprising parenterally administering to a patient in need thereof an effective dose of the conjugate of any one of claims 1, 24, 25 and 26.

38. A method for treating tumor sensitive to treatment with said method, said method comprising parenterally administering to a patient in need thereof an effective dose of the conjugate of claim 27.

39. A method for treating tumor sensitive to treatment with said method, said method comprising parenterally administering to a patient in need thereof an effective dose of the conjugate of claim 28.

40. A method for treating tumor sensitive to treatment with said method, said method comprising parenterally administering to a patient in need thereof an effective dose of the conjugate of claim 29.

41. The method of claim 37, wherein said tumor cells are selected from breast cancer cells, prostate cancer cells, ovarian cancer cells, colorectal cancer cells, gastric cancer cells, squamous cancer cells, small-cell lung cancer cells, testicular cancer cells, multiple myeloma cells, and cells from NK/T-cell malignancies.

42. The method of claim 37, wherein said tumor cells are selected from cells from NK/T-cell malignancies.

43. The method of claim 38, wherein said tumor cells are selected from breast cancer cells, prostate cancer cells, ovarian cancer cells, colorectal cancer cells, gastric cancer cells, squamous cancer cells, small-cell lung cancer cells, testicular cancer cells, multiple myeloma cells, and cells from NK/T-cell malignancies.

44. The method of claim 38, wherein said tumor cells are selected from cells from NK/T-cell malignancies.

45. The method of claim 39, wherein said tumor cells are selected from breast cancer cells, prostate cancer cells, ovarian cancer cells, colorectal cancer cells, gastric cancer cells, squamous cancer cells, small-cell lung cancer cells, testicular cancer cells, multiple myeloma cells, and cells from NK/T-cell malignancies.

46. The method of claim 39, wherein said tumor cells are selected from cells from NK/T-cell malignancies.

47. The method of claim 40, wherein said tumor cells are selected from breast cancer cells, prostate cancer cells, ovarian cancer cells, colorectal cancer cells, gastric cancer cells, squamous cancer cells, small-cell lung cancer cells, testicular cancer cells, multiple myeloma cells, and cells from NK/T-cell malignancies.

48. The method of claim 40, wherein said tumor cells are selected from cells from NK/T-cell malignancies.
